(19) United States
(12) Patent Application Publication
(10) Pub. No.: US 2024/0122732 A1
(43) Pub. Date: Apr. 18, 2024

(54) MULTI-BRANCH ENDOVASCULAR DEVICES AND METHODS

(71) Applicant: W. L. Gore & Associates, Inc., Newark, DE (US)

(72) Inventors: Merrill J. Birdno, Flagstaff, AZ (US); Jerald M. Crawley, Flagstaff, AZ (US); Kehinde A. Majolagbe, Flagstaff, AZ (US); Brandon C. Short, Flagstaff, AZ (US); Derek M. Ward, Flagstaff, AZ (US); Patrick S. Young, Flagstaff, AZ (US)

(21) Appl. No.: 18/278,211

(22) PCT Filed: Feb. 22, 2022
(86) PCT No.: PCT/US2022/017207

§ 371 Date: Aug. 22, 2023

Related U.S. Application Data
(60) Provisional application No. 63/152,164, filed on Feb. 22, 2021.

(51) Int. Cl. A61F 2/954; A61F 2/07

(52) U.S. Cl. CPC A61F 2/954; A61F 2/07; A61F 2002/061

(57) ABSTRACT

A multibranch implantable device including a main body including a tubular element having wall defining a main lumen, the tubular element having a first end defining a first opening into the main lumen and a second end defining a second opening in the main lumen, the tubular element including at least one side branch portal defining an aperture through the wall between the first longitudinal end and the second longitudinal end of the tubular element and at least one secondary body defining a secondary lumen, the at least one secondary body operable to be deployed with a portion of the secondary body positioned in the at least one side branch portal of the main body.

### CROSS-REFERENCE TO RELATED APPLICATION

This application is a national phase application of PCT Application No. PCT/US2022/017207, internationally filed on Feb. 22, 2022, which claims the benefit of Provisional Application No. 63/152,164, filed Feb. 22, 2021, which are incorporated herein by reference in their entireties for all purposes.

### FIELD

The present disclosure relates generally to endoluminal devices with multiple branches, and associated systems and methods. More specifically, the disclosure relates to endoluminal devices configured to be implemented in branched anatomical passageways.

### BACKGROUND

A variety of branched, anatomical passages may benefit from treatment in the form of an implanted, endoluminal device. One such passage is a vascular passage, such as an artery, with an aneurysm. Aneurysms occur in blood vessels at sites where, due to age, disease or genetic predisposition of the patient, the strength or resilience of the vessel wall is insufficient to prevent ballooning or stretching of the wall as blood passes through. If the aneurysm is left untreated, the blood vessel wall may expand and rupture, often resulting in death.

To prevent rupturing of an aneurysm, a stent graft may be introduced into a blood vessel percutaneously and deployed to span the aneurysmal sac. Various stent grafts include a graft fabric secured to a cylindrical scaffolding or framework of one or more stents. Generally, the stent(s) provide rigidity and structure to hold the graft open in a tubular configuration as well as the outward radial force needed to create a seal between the graft and a healthy portion of the vessel wall and provide migration resistance. Blood flowing through the vessel can be channeled through the luminal surface of the stent graft to reduce, if not eliminate, the stress on the vessel wall at the location of the aneurysmal sac. Stent grafts may reduce the risk of rupture of the blood vessel wall at the aneurysmal site and allow blood to flow through the vessel without interruption.

However, various endovascular repair procedures, such as the exclusion of an aneurysm, require a stent graft to be implanted adjacent to a vascular bifurcation. Often the aneurysm extends into the bifurcation requiring the stent graft to be placed into the bifurcation. A bifurcated stent graft is therefore required in these cases. Modular stent grafts, having a separate main body and branch component are often preferred in these procedures due to the ease and accuracy of deployment. See U.S. Patent Application No. 2008/0114446 to Hartley et al. for an example of a modular stent graft having separate main body and branch stent components. In the Hartley et al. publication the main body stent has a fenestration in the side wall that is tailored to engage and secure the side branch stent. The side branch stent in such a configuration is in a “line to line” interference fit with the main body fenestration, causing a potential compromise to the fatigue resistance of the stent to stent junction. U.S. Pat. No. 6,645,242 to Quinn presents a more robust stent to stent joining configuration. In the Quinn patent, a tubular support, internal to the main body stent, is incorporated to enhance the reliability of the stent to stent joining. The tubular, internal support of Quinn provides an extended sealing length along with improved fatigue resistance.

### SUMMARY

An endoprosthesis including a main body is provided with side branch portals for providing fluidic access to side branches of a main lumen when the main body of the endoprosthesis is deployed in the main lumen.

According to one example (“Example 1”), a multibranch implantable device includes a main body including a tubular element having wall defining a main lumen, the tubular element having a first end defining a first opening into the main lumen and a second end defining a second opening in the main lumen, the tubular element including at least one side branch portal defining an aperture through the wall between the first longitudinal end and the second longitudinal end of the tubular element; and at least one secondary body defining a secondary lumen, the at least one secondary body operable to be deployed with a portion of the secondary body positioned in the at least one side branch portal of the main body.

According to another example (“Example 2”), the multibranch device of Example 1, wherein the at least one side branch portal has a first end and a second end, the secondary body being operable to be deployed such that the second end of the side branch portal is substantially contiguous with an outer surface of the main body.

According to another example (“Example 3”), the multibranch device of any of the preceding Examples, wherein the first opening of the first longitudinal end of the tubular element faces a first direction and the second end of the at least one side branch portal is facing substantially in the first direction when the main body is in a neutral, unbent configuration.

According to another example (“Example 4”), the multibranch device of either Example 1 or Example 2, wherein the second opening of the second longitudinal end of the tubular element faces a second direction and the second end of the at least one side branch portal is facing substantially in the second direction when the main body is in a neutral, unbent configuration.

According to another example (“Example 5”), the multibranch device of any of the preceding Examples, wherein the wall of the main body defines a recess proximate the at least one side branch portal.

According to another example (“Example 6”), the multibranch device of any of the preceding Examples, wherein the main body further includes a stent coupled to the wall.

According to another example (“Example 7”), the multibranch device of any of the preceding Examples, wherein a portion of the wall defining the recess is unsupported.

According to another example (“Example 8”), the multibranch device of any of the preceding Examples, wherein the at least one side branch portal includes a first portal, a second portal, and a third portal each having exterior openings positioned at a first longitudinal position along the main body.

According to another example (“Example 9”), the multibranch device of any one of Examples 1-7, wherein the at least one side branch portal includes a first portal, a second portal, and a third portal each having an exterior opening, each exterior opening being positioned at one of at least two longitudinal positions along the main body.

According to another example (“Example 10”), a method of deploying an endoprosthesis at a target site having a main lumen and a first branch lumen includes advancing a main body of a multibranch stent graft toward the main lumen of the target site, the main body having a first portion and a second portion, the main body defining a first portal operable to provide fluidic access from the main body to a first side branch extending from the target site when the main body is deployed at the target site; partially deploying the first portion of the main body in the main lumen of the target site; advancing a first articulatable wire through the first portal and into the first branch lumen; partially deploying the second portion of the main body in the main lumen of the target site; fully deploying the first portion and the second portion of the main body; advancing a first side branch body along the first articulatable wire into the first branch lumen of the target site; and deploying the first side branch body in the first branch lumen of the target site.

According to another example (“Example 11”), the method of Example 10, wherein the first portal has a first end and a second end, the second end being substantially contiguous with an outer surface of the main body.

According to another example (“Example 12”), the method of either Example 10 or 11, wherein the main body defines a main body longitudinal axis and the first portal defines a first portal longitudinal axis, wherein the main body longitudinal axis and the first portal longitudinal axis are substantially parallel.

According to another example (“Example 13”), the method of any one of Examples 10-12, wherein the first portal is positioned such that the first portal from the first end to the second end is retrograde relative to fluid flow through the main body.

According to another example (“Example 14”), the method of any one of Examples 10-13, wherein walls of the main body defines a recess proximate the first portal such that when the first articulatable wire and the first side branch body are advanced, the recess provides clearance for the first articulatable wire and first side branch body to exit the first portal without kinking.

According to another example (“Example 15”), the method of any one of claim 10-14, wherein the main body includes a stent coupled to the wall and wherein a portion of the wall defining the recess does not include the stent.

According to another example (“Example 16”), the method of Example 10, wherein the main body includes a second portal and a third portal includes advancing a second articulatable wire through the second portal and into a second branch lumen of the target site; advancing a third articulatable wire through the third portal and into a third branch lumen of the target site; advancing a second side branch body along the second articulatable wire into the second branch lumen of the target site; and advancing a third side branch body along the third articulatable wire into the third branch lumen of the target site.

According to another example (“Example 17”), the method of Example 16, wherein the first side branch body is deployed prior to deploying the second side branch body and the third branch body.

According to another example (“Example 18”), the method of either Example 16 or 17, wherein exterior openings of each of the first portal, second portal, and third portal are each positioned at a first longitudinal position along the main body.

According to another example (“Example 19”), the method of either Example 16 or 17, wherein exterior openings of each of the first portal, second portal, and third portal each having an exterior opening, each exterior opening being positioned at one of at least two longitudinal positions along the main body.

According to another example (“Example 20”), the method of Example 10-19, further includes adjusting an inner curve of the main body prior to fully deploying the first portion and the second portion of the main body.

According to another example (“Example 11”), the method of Example 16, wherein each of the side branch bodies are deployed substantially simultaneously.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a side view of an implantable device with a main body and side branches, in accordance with an embodiment;

FIG. 2 is a side view of an implantable device deployed in a patient's aortic arch, in accordance with an embodiment;

FIG. 3 is a top view of a main body of an implantable device, the main body including side branch portals through which side branch members can be delivered and deployed, in accordance with an embodiment;

FIG. 4 is a side view of a main body of an implantable device, the main body including a portal access feature for providing clearance for side branch members delivered and deployed through the side branch portals in accordance with an embodiment;

FIG. 5 is an end view of a main body of an implantable device, the interior opening of the side branch portal positioned in the lumen of the main body, in accordance with one embodiment;

FIG. 6 is an end view of a main body of an implantable device, a portal access feature projecting into the lumen of the main body in accordance with one embodiment;

FIG. 7 is a top view of a main body of an implantable device, the main body including a stent structure that extends over side branch portals in accordance with one embodiment;

FIG. 8 is a perspective view of a main body including side branch portals staggered along a longitudinal length of the main body in accordance with one embodiment;

FIG. 9 is a perspective view of a main body including a side branch portal staggered relative to two side branch portals aligned along a longitudinal length of the main body in accordance with one embodiment;

FIG. 10 is a top view of a main body including side branch with a stent structure extending across a portal access feature in accordance with one embodiment;

FIG. 11 is a perspective view of a main body including a portal access feature in accordance with one embodiment;

FIG. 12 is a side view of a main body including a portal access feature in accordance with one embodiment;

FIG. 13 is an end view of a main body including a cartridge having side branch portals, the cartridge being deployable within a pocket of the main body in accordance with one embodiment;

FIG. 14 is an end view of a cartridge deployed within a pocket of a main body in accordance with one embodiment; and

FIG. 15 is a top view of a main body with a portal access feature supported by a portal access stent structure separate from the stent structure of the main body in accordance with one embodiment.

### DETAILED DESCRIPTION

### Definitions and Terminology

This disclosure is not meant to be read in a restrictive manner. For example, the terminology used in the application should be read broadly in the context of the meaning those in the field would attribute such terminology.

Persons skilled in the art will readily appreciate that various aspects of the present disclosure can be realized by any number of methods and apparatus configured to perform the intended functions. Stated differently, other methods and apparatus can be incorporated herein to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

Certain relative terminology is used to indicate the relative position of components and features. For example, words such as “top”, “bottom”, “upper,” “lower,” “left,” “right,” “horizontal,” “vertical,” “upward,” and “downward” are used in a relational sense (e.g., how components or features are positioned relative to one another) and not in an absolute sense unless context dictates otherwise. Similarly, throughout this disclosure, where a process or method is shown or described, the method may be performed in any order or simultaneously, unless it is clear from the context that the method depends on certain actions being performed first.

With respect to terminology of inexactitude, the terms “about” and “approximately” may be used, in certain instances, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example.

As used herein, “couple” means join, connect, attach, adhere, affix, or bond, whether directly or indirectly, and whether permanently or temporarily.

As used herein, the term “elastomer” refers to a polymer or a mixture of polymers that has the ability to be stretched to at least 1.3 times its original length and to retract rapidly to approximately its original length when released. The term “elastomeric material” refers to a polymer or a mixture of polymers that displays stretch and recovery properties similar to an elastomer, although not necessarily to the same degree of stretch and/or recovery. The term “non-elastomeric material” refers to a polymer or a mixture of polymers that displays stretch and recovery properties not similar to either an elastomer or elastomeric material, that is, considered not an elastomer or elastomeric material as is generally known.

The term “film” as used herein generically refers to one or more of the membrane, composite material, or laminate.

The term “biocompatible material” as used herein generically refers to any material with biocompatible characteristics including synthetic materials, such as, but not limited to, a biocompatible polymer, or a biological material, such as, but not limited to, bovine pericardium. Biocompatible material may comprise a first film and a second film as described herein for various embodiments.

For reference, the terms “circumference” and “diameter” are not meant to require a circular cross-section (although are inclusive of a circular cross-section), and are instead to be understood broadly to reference an outer surface or dimension and the dimension between opposing sides of the outer surface, respectively.

Although the embodiments herein may be described in connection with various principles and beliefs, the described embodiments should not be bound by theory. For example, embodiments are described herein in connection with vascular stent grafts, and more specifically branched stent grafts. However, embodiments within the scope of this disclosure can be applied toward any endoprostheses of similar structure and/or function. Furthermore, embodiments within the scope of this disclosure can be applied in non-vascular applications.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

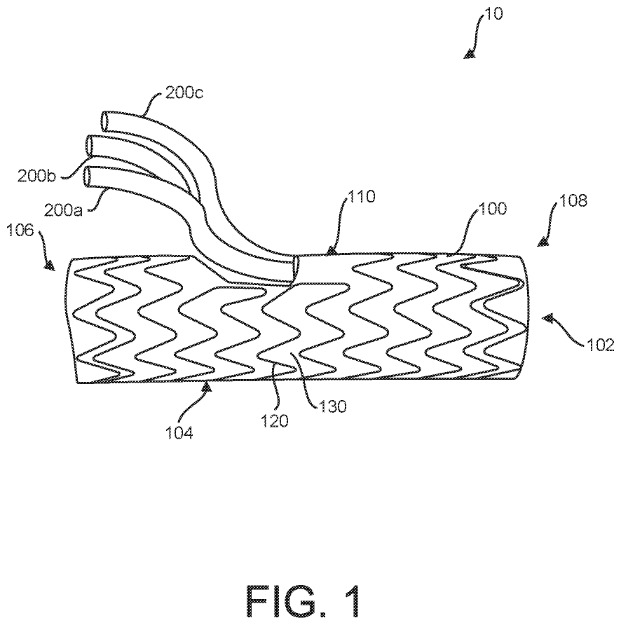

The device 10 shown in FIG. 1 is provided as an example of the various features of the device and, although the combination of those illustrated features is clearly within the scope of invention, that example and its illustration is not meant to suggest the inventive concepts provided herein are limited from fewer features, additional features, or alternative features to one or more of those features shown in FIG. 1.

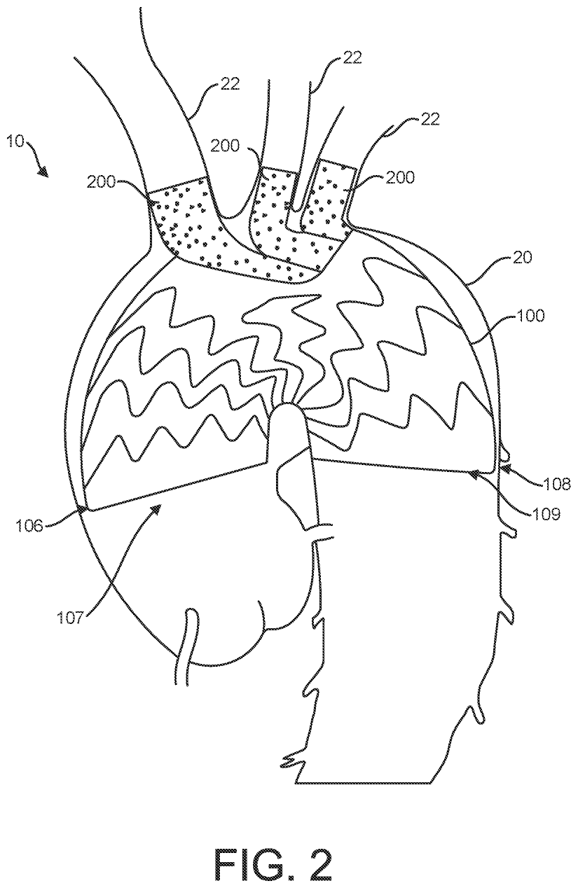

Referring to FIGS. 1 and 2, a device 10 is illustrated for treating disease along a main vessel 20 and at least one branch vessel 22 extending from the main vessel 20. The device 10 includes a main body 100 configured for deployment in the main vessel and having a main lumen 102. The device 10 also includes at least one branch member 200 for deployment in the at least one branch vessel and having a branch lumen 202.

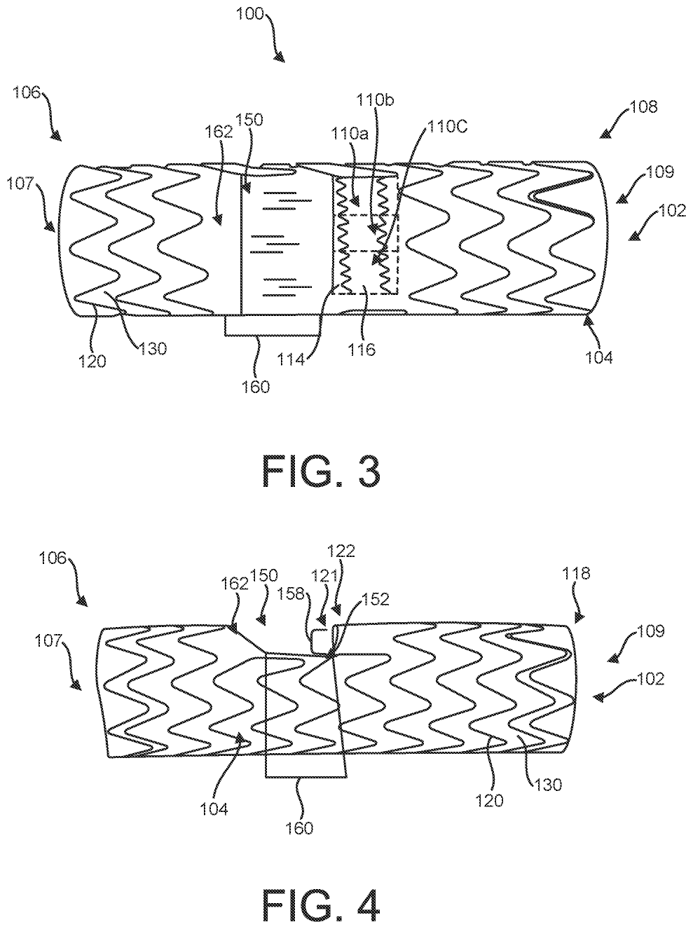

Referring to FIG. 3, an embodiment of the main body 100 is illustrated. The main body 100 includes a wall 104 forming the main lumen 102. The main body 100 has a first end 106 and a second end 108. At the first end 106, the main body 100 includes a first opening 107 and at the second end 108 the main body 100 includes a second opening 109. Each of the openings 107, 109 provides access to the main lumen 102 at the corresponding end 106, 108. Fluids may flow through the main lumen 102 by passing through the first opening 107, into the main lumen 102, and out the second opening 109, defining a main body fluid flow direction. Or, the flow may be in the opposite direction, defining the main body fluid flow direction. The outer wall 104 substantially forms or defines the outer profile of the main body 100.

In some embodiments, the main body 100 is formed of a stent structure 120 and a graft member 130. The stent structure 120 is operable to maintain patency of the main body 100 and/or the main vessel 20 when the main body 100 is deployed. The stent structure 120 can be formed of various materials, including, but not limited to, metals, metal alloys, polymers, and any combination thereof to provide elastic or plastic properties (e.g., self-expanding or balloon-expandable stents). The graft member 130 is coupled to the stent structure 120 and forms the fluid impermeable or semi-permeable layer through which fluids may flow (e.g., blood).

The main body 100 further includes at least one side branch portal 110. The side branch portal 110 is operable to provide fluidic access between the main lumen 102 and a branch vessel 22. The side branch portal 110 forms or is positioned in an opening 112 through the wall 104 along the outer profile of the main body 100. Stated otherwise, the side branch portal 110 extends through the wall 104 of the main body 100 longitudinally between the first end 106 and the second end 108 of the main body 100. Thus, fluid may flow through the first opening 107 and through the side branch portal 110. Some embodiments include a plurality of side branch portals 110. For example, FIG. 3 illustrates a main body 100 included a first side branch portal 110a, a second side branch portal 110b, and a third side branch portal 110c. Any number of side branch portals 110 may be incorporated to accommodate the specific anatomy into which the device 10 is to be deployed.

Referring still to FIG. 3, in some embodiments, each of the side branch portals 110 includes a side branch stent structure 114 and a side branch graft member 116. In various embodiments, the side branch stent structure 114 and side branch graft member 116 can be independent from, incorporated into, or integral with the main body stent structure 120 and the main body graft member 140. For example, as illustrated in FIGS. 3-5, the side branch stent structure 114 is separate or independent from the main body stent structure 120, whereas the side branch graft member 116 is incorporated into the main body graft member 140 (e.g., sandwiched or interposed between layers of the main body graft member 140). In some embodiments, the side branch stent structure 114 extends from the main body stent structure 120 and therefore represents a portion of the main body stent structure 120 rather than an independent stent structure. In still other embodiments, the side branch stent structure 114 is coupled to the main body stent structure 120. Similarly, the side branch graft member 116 can be formed directly from the main body graft member 140 and therefore represent a portion of the main body graft member 140. In other embodiments, the side branch graft member 116 is coupled to the main body graft member 140 or, or in still other embodiment, is spaced from the main body graft member 140. It is understood that any combination of side branch stent structures 114 and side branch graft member 116 embodiments is within the scope of this disclosure.

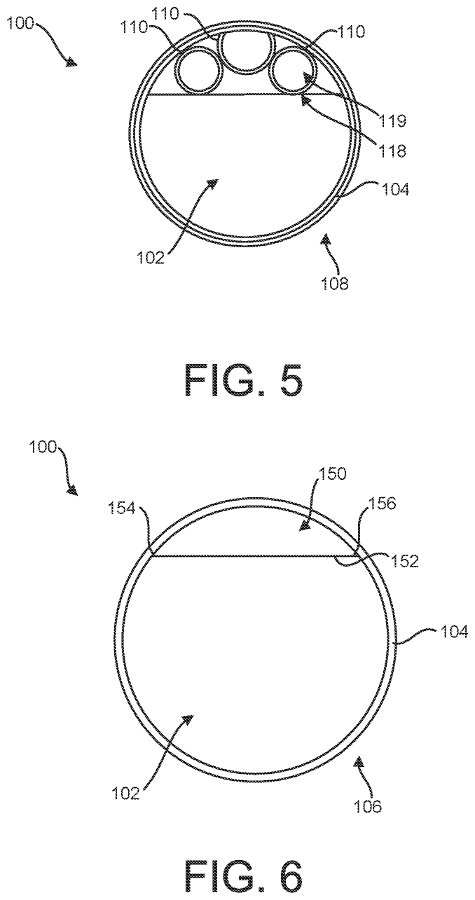

In some embodiments, the side branch portal 110 is positioned between the first end 106 and the second end 108 of the main body 100 and does not extend beyond or increase the outer profile of the main body 100 (see FIGS. 4 and 5). Stated otherwise, the portion of an outer wall of the side branch portal 110 is positioned along the wall 104 of the main body 100 within the outer profile of the main body 100 (e.g., flush with the outer profile). Thus, the side branch portal 110 may extend into the main lumen 102 of the main body 100 without substantially increasing the outer profile of the main body 100 adjacent the exit location of the side branch portal 110 from the main body 100.

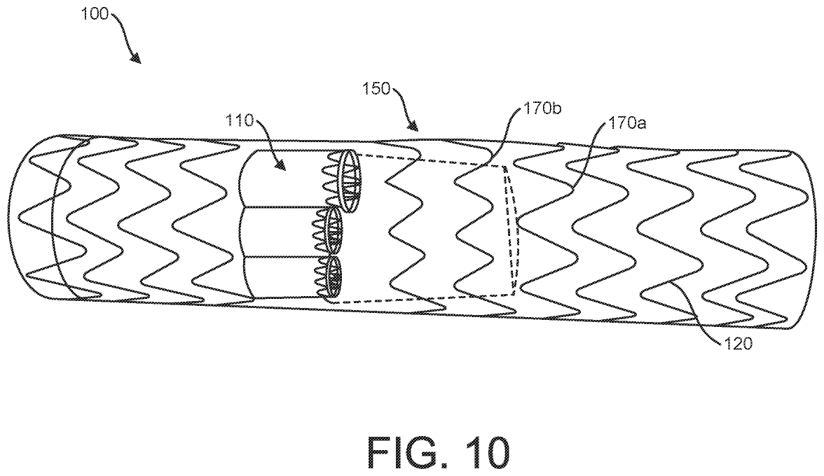

Each side branch portal 110 may be include a first end 118 and a second end 122 defining a first opening 119 and a second opening 121, respectively. Fluids travel through the side branch portal from the first end 118 to the second end 122 (or vice versa) defining a side branch fluid flow direction. The side branch portal 110 is positioned such that the first opening 119 is positioned within or oriented toward the main lumen 102 of the main body 100 and the second opening 121 is positioned exterior to or oriented away from the main body 100 (e.g., the first opening 119 is the interior opening and the second opening 121 is the exterior opening of the side branch portal 110 relative to the wall 104 and main lumen 102 of the main body 100). For example, FIG. 5 illustrates those embodiments in which the first opening 119 of the side branch portal 110 is positioned within the main lumen 102. The side branch portal 110 may have various longitudinal lengths. Furthermore, when a plurality of side branch portals 110 are implemented, one or more side branch portals 110 may have a different length than another of the side branch portals 110 or one or more of the side branch portals 110 may be have the same length as another of the side branch portals 110. In embodiments implementing a plurality of side branch portals 110, each side branch portal 110 may have a unique diameter and/or geometric orifice area (see FIG. 10) relative to the other side branch portals 110.

In some embodiments, the side branch portal 110 is oriented such that the side branch fluid flow direction is opposite to the main body fluid flow direction (e.g., retrograde to the main body fluid flow direction). It is understood that opposite or retrograde in these embodiments is not limited to 180 degrees of difference, but generally encompasses a change in the direction of the fluid flowing that is greater than 90 degrees. It is also understood that the direction of the fluid flow is with respect to the specific location along the longitudinal length of the main body 100 as the main body may conform to a curved anatomy. For example, in embodiments where the side branch fluid flow direction is opposite or retrograde to the main body fluid flow includes those embodiments in which the side branch portal 110 second opening 121 is longitudinally closer to the main body 100 first end 106 relative to the side branch portal 110 first opening 119. By orienting the side branch portal 110 in the retrograde orientation, a surgeon may be able to perform the intervention and any subsequent interventions from a more advantageous access site (e.g., femoral access site to reduce trauma to carotid arteries, subclavian, or other arteries or decrease surgical presence in more anatomically crowded portions of a patient such as around the neck or thorax when operating in the aortic arch). This orientation may be advantageous in some presentations where access may difficult, obstructed, or dangerous from certain access sites.

In other embodiments, the side branch portal 110 is oriented such that the side branch fluid flow direction is generally oriented with the main body fluid flow direction (e.g., antegrade to the main body fluid flow direction). In embodiments where the side branch fluid flow direction is antegrade to the main body fluid flow includes those embodiments in which the side branch portal 110 first opening 119 is longitudinally closer to the main body 100 first end 106 relative to the side branch portal 110 second opening 121. Antegrade orientations may be advantageous in some embodiments to maintain more traditional fluid flow, especially in tissues or anatomies that may have unique geometries that would limit the use of a retrograde orientation. In embodiments implementing a plurality of side branch portals 110, the side branch portal may all have an antegrade orientation, may all have a retrograde orientation, or may include one or more branch portals with an antegrade orientation and one or more portals having a retrograde orientation.

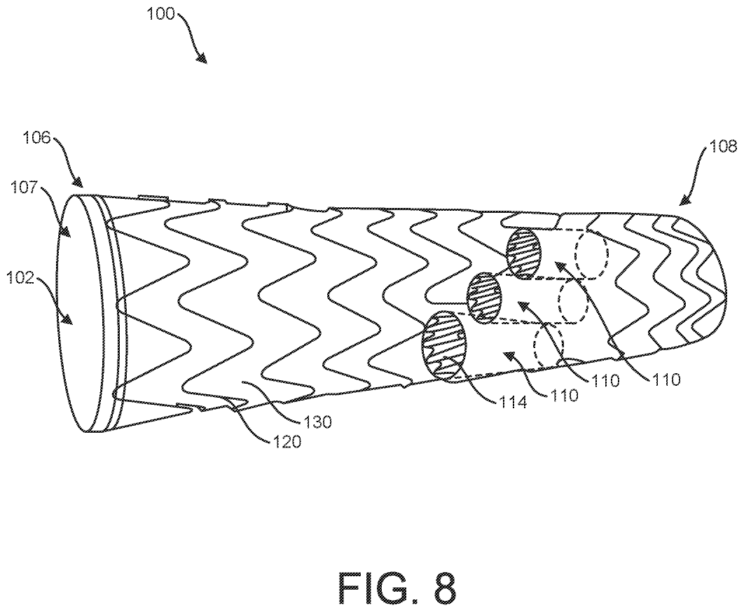

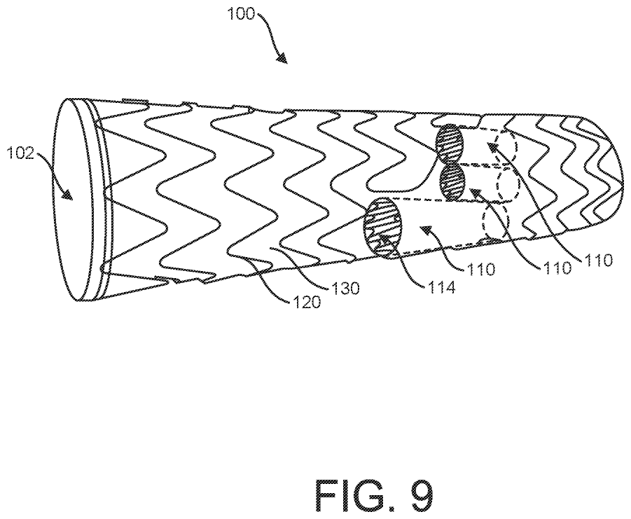

The second opening 121 of the side branch portal 110 can be positioned at various longitudinal positions between the first end 106 and the second end 108 of the main body 100. For example, the second opening 121 of the side branch portal 110 may be positioned generally at the midpoint between the first and second ends 106, 108 of the main body 100. In other embodiments, the second opening 121 of the side branch portal 110 may be positioned closer to the first end 106 relative the second end 108 or, alternatively, closer to the second end 108 relative to the first end 106 of the main body 100. In those embodiments including a plurality of side branch portals 110, each second opening 121 may be aligned longitudinally along the length of the main body 100 (see FIG. 3), staggered along the length of the main body 100 (see FIG. 8), or a combination thereof (see FIG. 9).

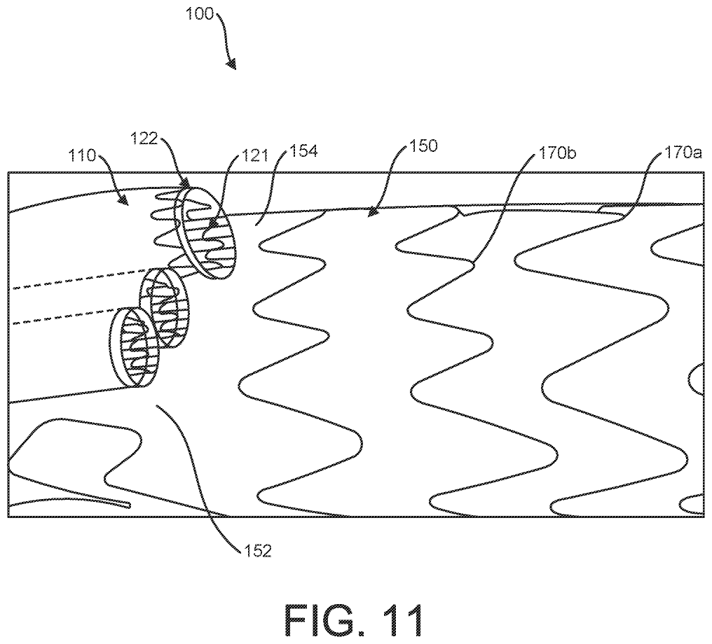

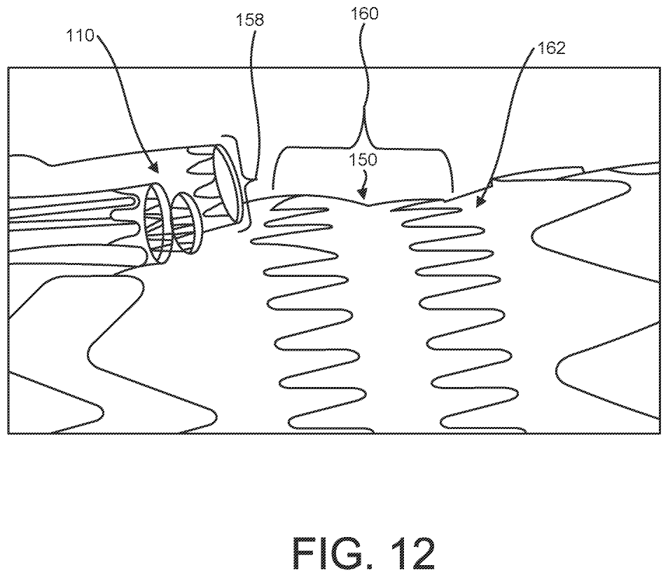

The side branch portals 110 may be incorporated into the main body 100 in variety of ways. For example, the side branch portals 110 may be wrapped between layers of film in the graft member 130 (e.g., FIG. 11). It is noted that in those embodiments in which a plurality of side branch portals 110 are implemented, a plug (not shown) may be inserted into any one or multiple side branch portals 110 if one or more of the side branch portals are not needed in a particular application. For example, a device 10 may include three side branch portals 110, but only two are needed for a patient (e.g., in the aortic arch with a bypass), one of the side branch portals 110 may be closed (e.g., via a plug).

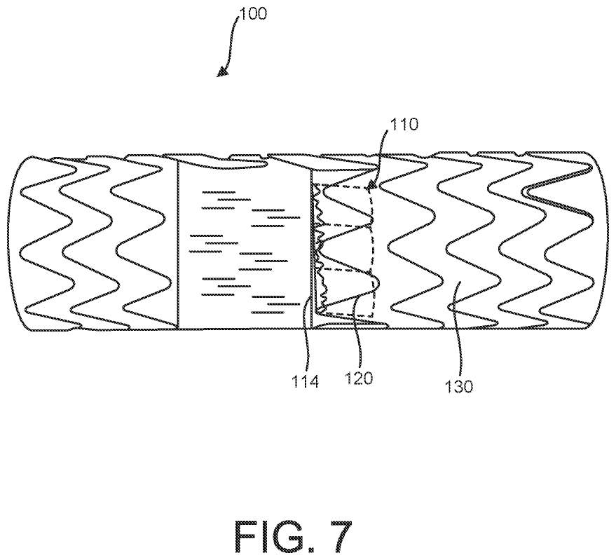

In some embodiments, the stent structure 120 extends around an outer periphery of the side branch portals 110 (FIG. 7). In embodiments implementing a side branch stent structure 114 that may implement materials that are more discreet or provide less holding or expansion force than the primary stent structure 120, the stent structure 120 may extend around the side branch portals 110 to limit collapsing of the side branch portals 110 and side branch stent structures 114 during delivery, deployment, and used of the device 10. However, in some embodiments, the stent structure 120 does not extend around the side branch portals 110 (see FIG. 4).

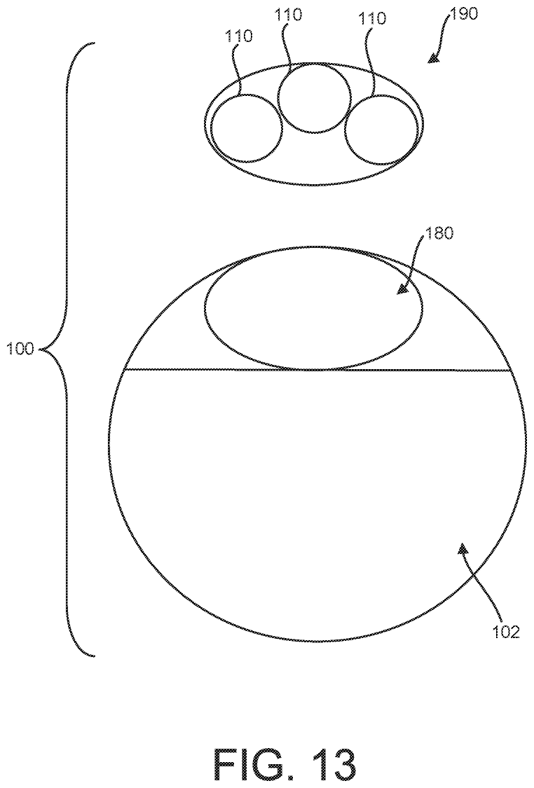

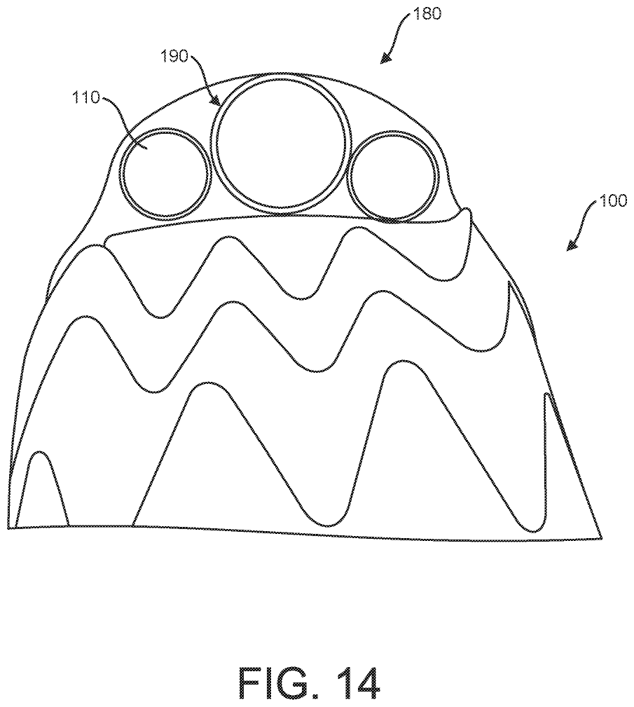

Referring to FIGS. 13 and 14, the main body 100 may form a pocket 180 into which the side branch portal 110 may be inserted. For example, in some embodiments, the side branch portals 110 are incorporated into a cartridge 190. The cartridge 190 is a modular element which can be inserted into the pocket 180 of the main body 100. For example, a cartridge may implement one, two, three, four, or any number of side branch portals 110 for use with the main body 100. This allows the device 10 to be customized to the specific needs of the patient. In some embodiments, the cartridge 190 includes one or more side branch portals 110 coupled together (e.g., via wrapping or film). The cartridge 190 provide access from outside of the main body to the main lumen 102 at a position between the first and second end 106, 108 via the side branch portals 110. This provides a modular solution to the customizing the device 10 with off the shelf components.

Referring now to FIG. 4, the main body 100 includes a portal access feature 150. The portal access feature 150 is operable to provide clearance for branch members 200 that are at least partially positioned and deployed within the side branch portal 110. For example, the portal access feature 150 may be a portion of the wall 104 of the main body 100 that has a recessed outer profile. For example, in FIG. 4, the main body 100 as illustrated includes a substantially circular cross-section along the longitudinal length of the main body 100 except at the longitudinal lengths of the main body 100 defining the portal access feature 150. FIG. 6 illustrates the main body 100 from a side view looking through the main lumen 102. In this view, the substantially circular outer profile is illustrated. This view also depicts the profile of the main body at the portal access feature 150. The main body 100 at the portal access feature 150 includes a cross-section that is substantially circular with a truncated or chord portion 152 of the wall 104 extending from a first position 154 of the wall 104 across to a second position 156 of the wall 104. As illustrated, the portal access feature 150 deviates from the typical outer profile of the remainder of the main body 100 such that the portal access feature 150 appears to be radially inward from the remainder of the main body 100.

Referring again to FIG. 4, the portal access feature 150 is defined in the wall 104 of the main body 100 from at least the second opening 121 of the side branch portal 110 toward the first end 106 of the main body. The depth 158 of the portal access feature 150 is substantially equal to diameter of the side branch portal 110. The portal access feature 150 may extend from the second opening 121 of the side branch portal 110 at the depth 158 for a predetermined length 160. The predetermined length 160 can provide sufficient space for the branch member 200 to exit the side branch portal 110 and turn or bend toward the branch vessel 22 and defines an entry portion 160 of the portal access feature 150. The entry portion 160 in some embodiments is substantially flat, as is illustrated in FIGS. 4 and 10. However the entry portion 160, in some embodiments, can incorporate a curvature. For example, in some embodiments, the entry portion 160 includes an arcuate profile. The arcuate profile may allow a plurality of side branch portals 110 to be implemented (e.g., each side branch portal 110 having the same diameter), where a bottom edge of each side branch portal 110 aligns with the entry portion 160 of the portal access feature 150 and the top edge aligns with the outer profile of the main body 100 (not shown). The portal access feature 150 may also include a transition portion 162. The transition portion 162 includes the portion of the wall 104 that transitions into the entry portion 160. The transition portion 162 may also be operable to accommodate the branch member 200 as it exits the side branch portal 110. In some embodiments, the transition portion 162 extends directly from the second opening 121 of the side branch portal 110 (not shown). In still further embodiments, the portal access feature 150 is a narrowing (not shown) of the main body 100 proximate the second opening 121 of the side branch portal 110.

It is understood that the portal access feature 150 does not have to begin at the second opening 121 of the side branch portal 110. For example, in some embodiments, the portal access feature 150 extends beneath the side branch portals 110. The side branch portals may be positioned between the portal access feature 150 and an outer layer of the graft member 130. In these embodiments, the portal access feature 150 extends from the side branch portal 110 toward the first end 106 of the main body 100.

With further reference to FIG. 4, the portal access feature 150, in some embodiments, the portal access feature is free of any stent. In some embodiments, the stent structure 120 used to support the graft member 130 does not extend onto the portal access feature 150. For example, in those embodiments in which the stent structure 120 is helically wound, the stent structure 120 does not extend across the portal access feature 150, but instead has a longitudinal portion 170 that extends along the length of the main body 100 proximate the portal access feature 150 and extends away from the portal access feature 150 at each end of the longitudinal portion 170. It is understood that the stent structure 120 can include various features such as apices 172, sinusoidal shapes and so forth while generally still being helically wound. In other embodiments, the stent structure 120 may include a plurality of independent rings that are longitudinally spaced along the length of the main body 100. The rings of the stent structure 120 that are positioned at a shared longitudinal length of the main body 100 with the portal access feature 150 may terminate proximate the portal access feature 150 instead of extending fully around the main body 100, or may include a longitudinal portion that connects rings as discussed with respect to helical winding.

In other embodiments, the stent structure 120 can extend across the portal access feature 150. For example, FIG. 10 illustrates an embodiment in which the stent structure 120 extends across the portal access feature 150, the stent structure being formed and/or shape set to accommodate and/or form the profile of the portal access feature 150. The portion of the stent structure 120 defined over the portal access feature 150 may be continuous with the remainder of the stent structure 120. For example, in main bodies 100 implementing a stent structure 120 that is helically disposed or wrapped about the main body 100, the stent structure 120 may substantially continue the helical path at the portal access feature 150. In some embodiments, the apices 170a of the stent structure 120 at the portal access feature 150 may be shorter than the apices 170b around the remainder of the main body 100 (see FIG. 11). Furthermore, the frequency may be decreased such that more apices are incorporated into a circumferential length of the main body 100 at the portal access feature 150. In other embodiments, the stent structure 120 positioned at the portal access feature 150 is shaped to outline or otherwise conform to the peripheral profile of the portal access feature 150. In these embodiments, the stent structure 120 of the portal access feature 150 extends from or is coupled to the stent structure 120 of the remainder of the main body 100, but has a shape independent from or not conforming to the pattern of the stent structure 120 of the remainder of the main body 100.

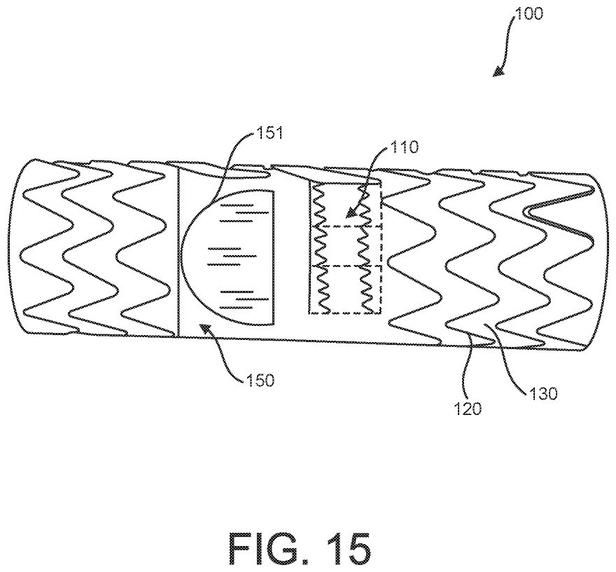

In some embodiments, the portal access feature 150 may include a portal access stent 151 (FIG. 15) that is independent from the stent structure 120 as previously discussed. The independent stent member can is coupled to the graft member 130 at the portal access feature 150. The independent stent member can incorporate any number of configurations, including patterns operable to conform to the peripheral profile of the portal access feature 150.

The portal access feature 150 may further include a reinforcing material. The reinforcing material is operable to provide increased strength to the portal access feature 150. The reinforcing material can resist tear, puncture, and other damage that can be incurred by the portal access feature 150 as the device 10 is being deployed. For example, cannulation and/or delivery and deployment of the branch member 200 may result in contacting the portal access feature, the reinforcing material being sufficiently sturdy to withstand tears or wear that could result in damage to the device 10. In some embodiments, the reinforcing material is applied to the portal access feature, is incorporated into the graft member 130 at the portal access feature, or a combination thereof. Various materials may be implemented for the reinforcement material, including but not limited to dense ePTFE layers or multilayers.

Referring to FIGS. 1 and 2, branch members 200 may be deployed through the side branch portals 110. The branch members 200 may include stents, stent grafts, grafts, and so forth. The stents and stent grafts may be self-expanding or balloon-expandable, for example. In one example, the device 10 can be deployed in the aortic arch. As shown, the device 10 includes a main body 100 with three side branch portals 110, although various numbers of side branch portals 110 are contemplated. In various embodiments, the first side branch portal 110a is operable to be cannulated for deployment of a first branch body 200a in the brachycephalic artery, a second side branch portal 110b with a second branch body 200b for the left common carotid artery, and a third side branch portal 110c with a third branch body 200c for the left subclavian artery.

The devices 10, including the main bodies 100 and branch members 200, described above may be made up of any material which is suitable for use as a graft or stent graft in the chosen body lumen. The grafts can be composed of the same or different materials. Furthermore, the grafts can comprise multiple layers of material that can be the same material or different material. In some examples, the graft can have several layers of material, including a layer that is formed into a tube (innermost tube) and an outermost layer that is formed into a tube (outermost tube).

A variety of material sets may be implemented for the graft members, including known vascular graft and stent graft materials. Polymers, biodegradable and natural materials can be used for specific applications. And, a variety of manufacturing techniques may be implemented to form the graft members, including extruding, coating, wrapping, combinations thereof, and others.

A biocompatible material for the graft components, discussed herein, may be used. In certain instances, the graft may include a fluoropolymer, such as a polytetrafluoroethylene (PTFE) polymer or an expanded polytetrafluoroethylene (ePTFE) polymer. In some instances, the graft may be formed of, such as, but not limited to, a polyester, a silicone, a urethane, a polyethylene terephthalate, or another biocompatible polymer, or combinations thereof. In some instances, bioresorbable or bioabsorbable materials may be used, for example a bioresorbable or bioabsorbable polymer. In some instances, the graft can include Dacron, polyolefins, carboxy methylcellulose fabrics, polyurethanes, or other woven, non-woven, or film elastomers.

Examples of suitable synthetic polymers include, but are not limited to nylon, polyacrylamide, polycarbonate, polyformaldehyde, polymethylmethacrylate, polytetrafluoroethylene, polytrifluorochlorethylene, polyvinylchloride, polyurethane, elastomeric organosilicon polymers, polyethylene, polypropylene, polyurethane, polyglycolic acid, polyesters, polyamides, their mixtures, blends and copolymers are suitable as a graft member. In one embodiment, the graft is made from a class of polyesters such as polyethylene terephthalate including DACRON® and MYLAR® and polyaramids such as KEVLAR®, polyfluorocarbons such as polytetrafluoroethylene (PTFE) with and without copolymerized hexafluoropropylene (TEFLON® or GORE-TEX®), and porous or nonporous polyurethanes. In another embodiment, the graft comprises expanded fluorocarbon polymers (especially PTFE) materials. Included in the class of preferred fluoropolymers are polytetrafluoroethylene (PTFE), fluorinated ethylene propylene (FEP), copolymers of tetrafluoroethylene (TFE) and perfluoro (propyl vinyl ether) (PFA), homopolymers of polychlorotrifluoroethylene (PCTFE), and its copolymers with TFE, ethylenechlorotrifluoroethylene (ECTFE), copolymers of ethylene-tetrafluoroethylene (ETFE), polyvinylidene fluoride (PVDF), and polyvinylfluoride (PVF). Especially preferred, because of its widespread use in vascular prostheses, is ePTFE. In another embodiment, the graft comprises a combination of the materials listed above. In another embodiment, the graft is substantially impermeable to bodily fluids. The substantially impermeable graft can be made from materials that are substantially impermeable to bodily fluids or can be constructed from permeable materials treated or manufactured to be substantially impermeable to bodily fluids (e.g. by layering different types of materials described above or known in the art). In one embodiment, the main body and branch members, as described above, are made from any combinations of the materials above. In another embodiment, the main body and branch members, as described above, comprise ePTFE.

The stents, as described above, may be generally cylindrical when restrained and/or when unrestrained and comprise helically arranged undulations having plurality of helical turns. The undulations preferably are aligned so that they are “in-phase” with each other. More specifically, undulations comprise apices in opposing first and second directions. When the undulations are in-phase, apices in adjacent helical turns are aligned so that apices can be displaced into respective apices of a corresponding undulation in an adjacent helical turn. In one embodiment, the undulations have a sinusoidal shape. In another embodiment, the undulations are U shaped. In another embodiment, the undulations are V shaped. In another embodiment, the undulations are ovaloid shaped.

In another embodiment, the stents, as described above, may also be provided in the form of a series of rings arranged generally coaxially along the graft body.

In various embodiments, the stent can be fabricated from a variety of biocompatible materials including commonly known materials (or combinations of materials) used in the manufacture of implantable medical devices. Typical materials include 316L stainless steel, cobalt-chromium-nickel-molybdenum iron alloy (“cobalt-chromium”), other cobalt alloys such as L605, tantalum, nitinol, or other biocompatible metals. In one embodiment, any stent-graft described herein is a balloon expandable stent-graft. In another embodiment, any stent-graft described herein is a self-expanding stent-graft. In another embodiment, the stent is a wire wound stent. In another embodiment, the wire wound stent includes undulations, or a repeating, undulating pattern of apices.

The wire wound stent can be constructed from a reasonably high strength material, e.g., one which is resistant to plastic deformation when stressed. In one embodiment, the stent member comprises a wire which is helically wound around a mandrel having pins arranged thereon so that the helical turns and undulations can be formed simultaneously, as described below. Other constructions also may be used. For example, an appropriate shape may be formed from a flat stock and wound into a cylinder or a length of tubing formed into an appropriate shape or laser cutting a sheet of material. In another embodiment, said stent is made from a super-elastic alloy. There are a variety of disclosures in which super-elastic alloys such as nitinol are used in stents.

A variety of materials variously metallic, super elastic alloys, such as Nitinol, are suitable for use in these stents. Primary requirements of the materials are that they be suitably springy even when fashioned into very thin sheets or small diameter wires. Various stainless steels which have been physically, chemically, and otherwise treated to produce high springiness are suitable as are other metal alloys such as cobalt chrome alloys (e.g., ELGILOY®), platinum/tungsten alloys, and especially the nickel-titanium alloys generically known as “nitinol”.

Nitinol is especially preferred because of its “super-elastic” or “pseudo-elastic” shape recovery properties, i.e., the ability to withstand a significant amount of bending and flexing and yet return to its original form without permanent deformation. These metals are characterized by their ability to be transformed from an austenitic crystal structure to a stress-induced martensitic structure at certain temperatures, and to return elastically to the austenitic shape when the stress is released. These alternating crystalline structures provide the alloy with its super-elastic properties.

Other suitable stent materials include certain polymeric materials, particularly engineering plastics such as thermotropic liquid crystal polymers (“LCP's”). These polymers are high molecular weight materials which can exist in a so-called “liquid crystalline state” where the material has some of the properties of a liquid (in that it can flow) but retains the long range molecular order of a crystal. The term “thermotropic” refers to the class of LCP's which are formed by temperature adjustment. LCP's may be prepared from monomers such as p,p′-dihydroxy-polynuclear-aromatics or dicarboxy-polynuclear-aromatics. The LCP's are easily formed and retain the necessary interpolymer attraction at room temperature to act as high strength plastic artifacts as are needed as a foldable stent. They are particularly suitable when augmented or filled with fibers such as those of the metals or alloys discussed below. It is to be noted that the fibers need not be linear but may have some preforming such as corrugations which add to the physical torsion enhancing abilities of the composite.

Any of a variety of bio-active agents may be implemented with any of the foregoing. For example, any one or more of (including portions thereof) the device 10 may comprise a bio-active agent. Bio-active agents can be coated onto one or more of the foregoing features for controlled release of the agents once the device 10 is implanted. Such bio-active agents can include, but are not limited to, thrombogenic agents such as, but not limited to, heparin. Bio-active agents can also include, but are not limited to agents such as anti-proliferative/antimitotic agents including natural products such as vinca alkaloids (e.g., vinblastine, vincristine, and vinorelbine), paclitaxel, epidipodophyllotoxins (e.g., etoposide and teniposide), antibiotics (e.g., dactinomycin (actinomycin D), daunorubicin, doxorubicin, and idarubicin), anthracyclines, mitoxantrone, bleomycins, plicamycin (mithramycin) and mitomycin, enzymes (e.g., L-asparaginase which systemically metabolizes L-asparagine and deprives cells which do not have the capacity to synthesize their own asparagine); antiplatelet agents such as G(GP) IIb/IIIa inhibitors and vitronectin receptor antagonists; anti-proliferative/antimitotic alkylating agents such as nitrogen mustards (e.g., mechlorethamine, cyclophosphamide and analogs, melphalan, chlorambucil), ethylenimines and methylmelamines (e.g., hexamethylmelamine and thiotepa), alkyl sulfonates-busulfan, nitrosoureas (e.g., carmustine (BCNU) and analogs, streptozocin), trazenes-dacarbazinine (DTIC); anti-proliferative/antimitotic antimetabolites such as folic acid analogs (e.g., methotrexate), pyrimidine analogs (e.g., fluorouracil, floxuridine, and cytarabine), purine analogs and related inhibitors (e.g., mercaptopurine, thioguanine, pentostatin and 2-chlorodeoxyadenosine {cladribine}); platinum coordination complexes (e.g., cisplatin and carboplatin), procarbazine, hydroxyurea, mitotane, am inoglutethimide; hormones (e.g., estrogen); anti-coagulants (e.g., heparin, synthetic heparin salts and other inhibitors of thrombin); anti-platelet agents (e.g., aspirin, clopidogrel, prasugrel, and ticagrelor); vasodilators (e.g., heparin, aspirin); fibrinolytic agents (e.g., plasminogen activator, streptokinase, and urokinase), aspirin, dipyridamole, ticlopidine, clopidogrel, abciximab; antimigratory agents; antisecretory agents (e.g., breveldin); anti-inflammatory agents, such as adrenocortical steroids (e.g., cortisol, cortisone, fludrocortisone, prednisone, prednisolone, 6α-methylprednisolone, triamcinolone, betamethasone, and dexamethasone), non-steroidal agents (e.g., salicylic acid derivatives, such as aspirin); para-aminophenol derivatives (e.g., acetaminophen); indole and indene acetic acids (e.g., indomethacin, sulindac, and etodalac), heteroaryl acetic acids (e.g., tolmetin, diclofenac, and ketorolac), arylpropionic acids (e.g., ibuprofen and derivatives), anthranilic acids (e.g., mefenamic acid and meclofenamic acid), enolic acids (e.g., piroxicam, tenoxicam, phenylbutazone, and oxyphenthatrazone), nabumetone, gold compounds (e.g., auranofin, aurothioglucose, and gold sodium thiomalate); immunosuppressives (e.g., cyclosporine, tacrolimus (FK-506), sirolimus (rapamycin), azathioprine, and mycophenolate mofetil); angiogenic agents (e.g., vascular endothelial growth factor (VEGF)), fibroblast growth factor (FGF); angiotensin receptor blockers; nitric oxide donors; anti-sense oligonucleotides and combinations thereof; cell cycle inhibitors, mTOR inhibitors, growth factor receptor signal transduction kinase inhibitors; retinoids; cyclin/CDK inhibitors; HMG co-enzyme reductase inhibitors (statins); and protease inhibitors.

Various methods of deploying the device 10 may be implemented. For example, a method of deploying an endoprosthesis at a target site having a main lumen and a first branch lumen (e.g., aortic arch with the brachiocephalic artery, left common carotid artery, and the left subclavian artery), may include the following: (1) advancing a main body of a multibranch stent graft toward the main lumen of the target site, the main body having a first portion and a second portion, the main body defining a first portal operable to provide fluidic access from the main body to a first side branch extending from the target site when the main body is deployed at the target site; (2) partially deploying the first portion of the main body in the main lumen of the target site; (3) advancing a first articulatable wire through the first portal and into the first branch lumen; (4) partially deploying the second portion of the main body in the main lumen of the target site; (5) fully deploying the first portion and the second portion of the main body; (6) advancing a first side branch body along the first articulatable wire into the first branch lumen of the target site; and (7) deploying the first side branch body in the first branch lumen of the target site.

In some embodiments, the first portal has a first end and a second end, the second end being substantially contiguous with an outer surface of the main body. This maintains an outer profile of the device for conformation with the surrounding anatomy. The main body may define a main body longitudinal axis and the first portal may define a first portal longitudinal axis, wherein the main body longitudinal axis and the first portal longitudinal axis are substantially parallel. In some embodiments, the first portal is positioned such that the first portal from the first end to the second end is retrograde relative to fluid flow through the main body. The walls of the main body may define a recess proximate the first portal such that when the first articulatable wire and the first side branch body are advanced, the recess provides clearance for the first articulatable wire and first side branch body to exit the portal without kinking. In some embodiments, the main body includes a stent coupled to the wall and wherein a portion of the wall defining the recess does not include the stent.

In embodiments where the main body includes a second portal and a third portal method of claim 10, the method may further include: (1) advancing a second articulatable wire through the second portal and into a second branch lumen of the target site; (2) advancing a third articulatable wire through the third portal and into a third branch lumen of the target site; (3) advancing a second side branch body along the second articulatable wire into the second branch lumen of the target site; and (4) advancing a third side branch body along the third articulatable wire into the third branch lumen of the target site. In these embodiments, the first side branch body may be deployed prior to deploying the second side branch body and the third branch body. Various arrangements of the portals may be implemented, including where exterior openings of each of the first portal, second portal, and third portal are each positioned at a first longitudinal position along the main body, or where exterior openings of each of the first portal, second portal, and third portal are each positioned at different longitudinal positions along the main body. In some embodiments, the method further includes adjusting an inner curve of the main body prior to fully deploying the first portion and the second portion of the main body.

Numerous characteristics and advantages of the present invention have been set forth in the preceding description, including preferred and alternate embodiments together with details of the structure and function of the invention. The disclosure is intended as illustrative only and as such is not intended to be exhaustive. It will be evident to those skilled in the art that various modifications may be made, especially in matters of structure, materials, elements, components, shape, size and arrangement of parts within the principals of the invention, to the full extent indicated by the broad, general meaning of the terms in which the appended claims are expressed. To the extent that these various modifications do not depart from the spirit and scope of the appended claims, they are intended to be encompassed therein. In addition to being directed to the embodiments described above and claimed below, the present invention is further directed to embodiments having different combinations of the features described above and claimed below.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A multibranch implantable device comprising:
a main body including a tubular element having wall defining a main lumen, the tubular element having a first end defining a first opening into the main lumen and a second end defining a second opening in the main lumen, the tubular element including at least one side branch portal defining an aperture through the wall between the first longitudinal end and the second longitudinal end of the tubular element; and
at least one secondary body defining a secondary lumen, the at least one secondary body operable to be deployed with a portion of the secondary body positioned in the at least one side branch portal of the main body.

2. The multibranch device of claim 1, wherein the at least one side branch portal has a first end and a second end, the secondary body being operable to be deployed such that the second end of the side branch portal is substantially contiguous with an outer surface of the main body.

3. The multibranch device of claim 1, wherein the first opening of the first longitudinal end of the tubular element faces a first direction and the second end of the at least one side branch portal is facing substantially in the first direction when the main body is in a neutral, unbent configuration.

4. The multibranch device of either claim 1, wherein the second opening of the second longitudinal end of the tubular element faces a second direction and the second end of the at least one side branch portal is facing substantially in the second direction when the main body is in a neutral, unbent configuration.

5. The multibranch device of claim 1, wherein the wall of the main body defines a recess proximate the at least one side branch portal.

6. The multibranch device of claim 1, wherein the main body further includes a stent coupled to the wall.

7. The multibranch device of claim 1, wherein a portion of the wall defining the recess is unsupported.

8. The multibranch device of claim 1, wherein the at least one side branch portal includes a first portal, a second portal, and a third portal each having exterior openings positioned at a first longitudinal position along the main body.

9. The multibranch device of claim 1, wherein the at least one side branch portal includes a first portal, a second portal, and a third portal each having an exterior opening, each exterior opening being positioned at one of at least two longitudinal positions along the main body.

10. A method of deploying an endoprosthesis at a target site having a main lumen and a first branch lumen, the method comprising:
advancing a main body of a multibranch stent graft toward the main lumen of the target site, the main body having a first portion and a second portion, the main body defining a first portal operable to provide fluidic access from the main body to a first side branch extending from the target site when the main body is deployed at the target site;
partially deploying the first portion of the main body in the main lumen of the target site;
advancing a first articulatable wire through the first portal and into the first branch lumen;
partially deploying the second portion of the main body in the main lumen of the target site;
fully deploying the first portion and the second portion of the main body;
advancing a first side branch body along the first articulatable wire into the first branch lumen of the target site; and
deploying the first side branch body in the first branch lumen of the target site.

11. The method of claim 10, wherein the first portal has a first end and a second end, the second end being substantially contiguous with an outer surface of the main body.

12. The method of claim 10, wherein the main body defines a main body longitudinal axis and the first portal defines a first portal longitudinal axis, wherein the main body longitudinal axis and the first portal longitudinal axis are substantially parallel.

13. The method of claim 10, wherein the first portal is positioned such that the first portal from the first end to the second end is retrograde relative to fluid flow through the main body.

14. The method of claim 10, wherein walls of the main body defines a recess proximate the first portal such that when the first articulatable wire and the first side branch body are advanced, the recess provides clearance for the first articulatable wire and first side branch body to exit the first portal without kinking.

15. The method of claim 10, wherein the main body includes a stent coupled to the wall and wherein a portion of the wall defining the recess does not include the stent.

16. The method of claim 10, wherein the main body includes a second portal and a third portal, the method further comprising:
advancing a second articulatable wire through the second portal and into a second branch lumen of the target site;
advancing a third articulatable wire through the third portal and into a third branch lumen of the target site;
advancing a second side branch body along the second articulatable wire into the second branch lumen of the target site; and
advancing a third side branch body along the third articulatable wire into the third branch lumen of the target site.

17. The method of claim 16, wherein the first side branch body is deployed prior to deploying the second side branch body and the third branch body.

18. The method of claim 16, wherein exterior openings of each of the first portal, second portal, and third portal are each positioned at a first longitudinal position along the main body.

19. The method of claim 16, wherein exterior openings of each of the first portal, second portal, and third portal each having an exterior opening, each exterior opening being positioned at one of at least two longitudinal positions along the main body.

20. The method of claim 10, further comprising adjusting an inner curve of the main body prior to fully deploying the first portion and the second portion of the main body.

21. The method of claim 16, wherein each of the side branch bodies are deployed substantially simultaneously.
